(19) United States
(12) Patent Application Publication
(10) Pub. No.: US 2024/0294859 A1
(43) Pub. Date: Sep. 5, 2024

(54) MULTI-LEVEL PORT VESSEL FOR USE IN BIOREACTOR SYSTEMS

(71) Applicant: PBS BIOTECH, INC., Camarillo, CA (US)

(72) Inventors: Gary Evans, Macatawa, MI (US); Sunghoon Jung, Camarillo, CA (US)

(21) Appl. No.: 18/589,775

(22) Filed: Feb. 28, 2024

Related U.S. Application Data
(60) Provisional application No. 63/487,699, filed on Mar. 1, 2023.

(51) Int. Cl. C12M 1/00; C12M 1/06; C12M 1/26

(52) U.S. Cl. CPC C12M 29/00; C12M 23/38; C12M 27/06; C12M 33/04

(57) ABSTRACT

A multi-level port vessel includes a vessel body defining an interior volume. The vessel body has a front wall, a rear wall, two side walls, a bottom, and a top. The top is open and adapted to be covered by a removable lid. A vertically oriented mixing wheel is disposed within the interior volume. The vertically oriented mixing wheel has a substantially horizontal rotation axis when the vessel body is disposed on a substantially horizontal surface. A plurality of ports is disposed in one of the rear wall, the front wall, or the side walls. At least one port in the plurality of ports is located at an elevation that is at least partially encompassed by a projection of the perimeter of the vertically oriented mixing wheel.

### CROSS-REFERENCE TO RELATED APPLICATIONS

Priority is claimed to U.S. Provisional Patent Application No. 63/487,699, filed Mar. 1, 2023, the entire contents of which are hereby expressly incorporated by reference herein.

### FIELD OF THE DISCLOSURE

This disclosure relates generally to a vessel and, more specifically, to a vessel having multi-level ports.

### BACKGROUND

The medical and pharmaceutical industries use living cells to produce biologically beneficial products such as antibodies, cytokines, viral gene vectors, or biochemically active substances derived from the living cells. Bioreactor systems support the biologically active environments needed to produce the biologically beneficial products listed above. Bioreactor systems generally include a vessel, a tank or a housing (often referred to as a reactor tank or a reactor vessel) that encloses an internal volume, a medium input for delivering a medium (such as a liquid carrying living cells) to the internal volume, and an agitator to maintain a homogeneous environment within the internal volume. Bioreactor systems can optionally include one or more of aerators, thermal jackets, sensors, and effluent outlets, depending upon the needs of the system.

Traditionally, the agitators took the form of a horizontal impeller or other stirring rod that is rotated by a vertically oriented shaft connected to a motor. Other agitators included tilting tables, upon which the tank or housing is placed to slowly slosh the liquid in the tank back and forth, thereby maintaining a homogeneous mixture. In either case, sensors, or sample ports are oriented vertically, and inserted from a top of the reactor vessel. The sensors or sample ports must not interfere with the agitator, so in reactor vessels with impellers or stirring rods, the sample pipettes or the sensors were limited to certain elevations in the tank above the impeller or stirring rods.

Recently, bioreactor systems having vessels with vertically oriented mixing wheels having a horizontally oriented rotational axis, have been developed. While these vertically oriented mixing wheels offer superior mixing within the reactor vessel, for example by lowering shear stress and/or providing more complete mixing, sensors and sample ports are still located at the top of the reactor vessel. As a result, only certain locations within the reactor vessel are accessible as the vertically oriented mixing wheel blocks a significant portion of the reactor vessel from access.

Turning now to FIG. 1, a prior art example of a vessel 10 for use in a bioreactor system, where the vessel includes a vertically oriented mixing wheel, is illustrated. In some versions, the vessel can include a single-use vessel. The vessel 10 includes a vessel body 20 defined by outer walls 21 that hold cells and excipient. A vertically oriented mixing wheel 22 is enclosed within the vessel body 20 for mixing and maintaining cells in suspension. A vertically oriented wheel is a wheel having a horizontal rotation axis so that the wheel rotates in a vertical plane when the vessel is placed on a horizontal surface. The outer walls 21 include a lower curved wall 23. The vertically oriented wheel 22 is positioned in a lower portion of the containment vessel and oriented in a vertical plane and rotates about a horizontal axis 24. Cells and excipient are introduced into the vessel by removing a threaded port cap 26 via pipetting or pouring, for example. The cap 26 may be threaded back onto the port to seal prior to cell dispensing to minimize the potential for introducing foreign materials. A hydrophobic membrane 28 on the cap 26 allows improved thermal exchange with the air in the local environment to help maintain temperature.

During cell dispensing, in some bioreactors, fluid can be removed at a lower dispenser 30 via a vessel orifice 32 that extends through an outer wall near the bottom of the vessel body 20. The fluid travels down a bore in a machined block 36 of the dispenser 30 which is affixed to the vessel body 20 and sealed around the orifice 32. A hose barb adaptor 38 opens to the bore that mates with the machined block 36 and allows tubing to be secured to it to maintain a sterile fluid path.

The vertically oriented wheel 22 includes of a plurality of paddles 40 along its outer periphery that generate a sweeping motion of the liquid during rotation to counteract cell settling in the excipient. The paddles 40, may include permanent magnets 41, which are used to couple with magnets on an agitation controller (not shown) to drive the rotation of the vertically oriented wheel 22. The vertically oriented wheel 22 may also include two diametrically-opposed vanes 42 extending from the paddles 40 to an inner hub that create bi-axial fluid flow as the impeller rotates.

### SUMMARY OF THE DISCLOSURE

In a first example, the present disclosure relates to a multi-level port vessel including a vessel body defining an interior volume. The vessel body has a front wall, a rear wall, two side walls, a bottom, and a top. The top is open and adapted to be covered by a removable lid. A vertically oriented mixing wheel is disposed within the interior volume. The vertically oriented mixing wheel has a substantially horizontal rotation axis when the vessel body is disposed on a substantially horizontal surface. A plurality of ports is disposed in one of the rear wall, the front wall, or the side walls. At least one port in the plurality of ports is located at an elevation that is at least partially encompassed by a projection of the perimeter of the vertically oriented mixing wheel.

The above example of a vessel may further include any one or more of the following optional forms.

In one optional form, the plurality of ports comprises two or more ports.

In another optional form, the plurality of ports comprises a first port located at an elevation at least partially above the projection of the perimeter of the vertically oriented mixing wheel. A second port is located at an elevation that is at least partially encompassed by the projection of the perimeter of the vertically oriented mixing wheel. A third port is located at an elevation at least partially below the projection of the perimeter of the vertically oriented mixing wheel.

In another optional form, the first port is positioned at a first height above the second port, and the first height corresponds to a volume of at least 66% of a full usable volume of the interior volume of the vessel body as measured from the bottom of the vessel body.

In another optional form, the second port is positioned at a second height above the third port, and the second height corresponds to a volume of at least 12% of a full usable volume of the interior volume of the vessel body as measured from the bottom of the vessel body.

In another optional form, a tube guide is located proximate to at least one port.

In another optional form, the tube guide includes a pair of outwardly extending ribs that are configured to receive a tube that is connected to at least one port for providing access to the interior volume via at least one port.

In another optional form, a plurality of tube guides are disposed proximate to the ports, each tube guide in the plurality of tube guides being located proximate to a different port in the plurality of ports.

In another optional form, at least one port is fluidly connected to a molded elbow, which is secured to the tube. The molded elbow may have an inlet and an outlet and the outlet including a barbed end for securing to the tube.

In another optional form, an o-ring is disposed between the molded elbow and the port.

In another optional form, the molded elbow comprises an angled cone inlet so that a portion of the angled cone is not perpendicular to the port.

In another optional form, a removable lid covers the top of the vessel.

In another optional form, the removable lid comprises a cut-out portion having at least one recess configured to receive a tube connected to at least one port.

In another optional form, the removable lid comprises at least one inlet opening.

In another optional form, the vertically oriented mixing wheel comprises a plurality of mixing paddles.

In another optional form, the vertically oriented mixing wheel comprises a plurality of magnets.

In another optional form, at least one port is located at the rear wall of the vessel.

In another optional form, another port is located at one of the side walls of the vessel.

In a second example, the present disclosure relates to a method of accessing an interior of a vessel. The method can include providing a vessel defining an interior volume, the vessel including a mixing wheel. The method can further include accessing the interior volume of the vessel via one of a plurality of ports in the vessel, each port being located at one of a rear wall, a front wall, or a side wall of the vessel, wherein at least one of the plurality of ports is located at an elevation that is at least partially occupied by a projection of the perimeter of the mixing wheel.

The above example of a method of extracting fluid from a vessel may further include any one or more of the following optional forms.

In one optional form, accessing the interior volume includes extracting fluid from the interior volume via the port.

In another optional form, accessing the interior volume includes penetrating the port with an extraction tool.

In yet another optional form, accessing the interior volume includes adding fluid, gas, cells, buffer, and/or other media to the interior volume of the vessel through the port.

In yet another optional form, accessing the interior volume includes penetrating the port with an introducer tool.

In yet another optional form, accessing the interior volume includes passing a sensor device through the port and into the interior volume of the vessel.

In yet another optional form, accessing the interior volume includes rotating the mixing wheel, and accessing the interior volume of the vessel while the mixing wheel is rotating.

In yet another optional form, wherein accessing the interior volume of the vessel includes accessing the interior volume along a horizontal axis.

In yet another optional form, accessing the interior volume of the vessel comprises accessing the interior volume at a location that is at an elevation that is (i) occupied at least partially by a projection of the perimeter of the mixing wheel, (ii) at least partially above a projection of the perimeter of the mixing wheel, or (iii) at least partially below a projection of the perimeter of the mixing wheel.

### BRIEF DESCRIPTION OF THE DRAWINGS

FIG. 1 is a perspective view of a prior-art vessel having a vertically oriented wheel;

FIG. 2 is a perspective view of a vessel having multi-level ports constructed in accordance with the disclosure;

FIG. 3 is a front view of the vessel of FIG. 2;

FIG. 4 is a rear view of the vessel of FIG. 2;

FIG. 5 is a right side view of the vessel of FIG. 2;

FIG. 6 is a left side view of the vessel of FIG. 2;

FIG. 7 is a close up lower right side view of the vessel of FIG. 2 showing a lower port; and

FIG. 8 is a close up view of a port of FIG. 7.

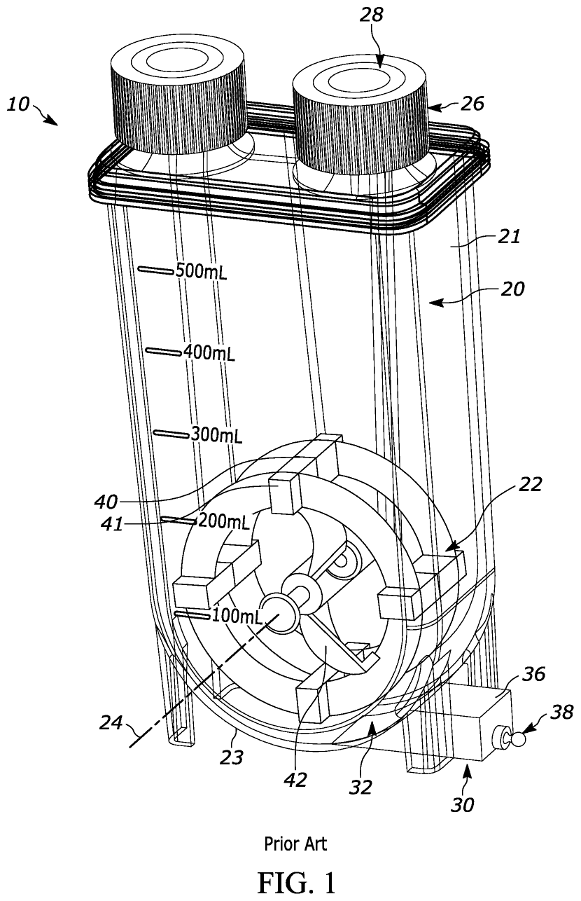

### DETAILED DESCRIPTION

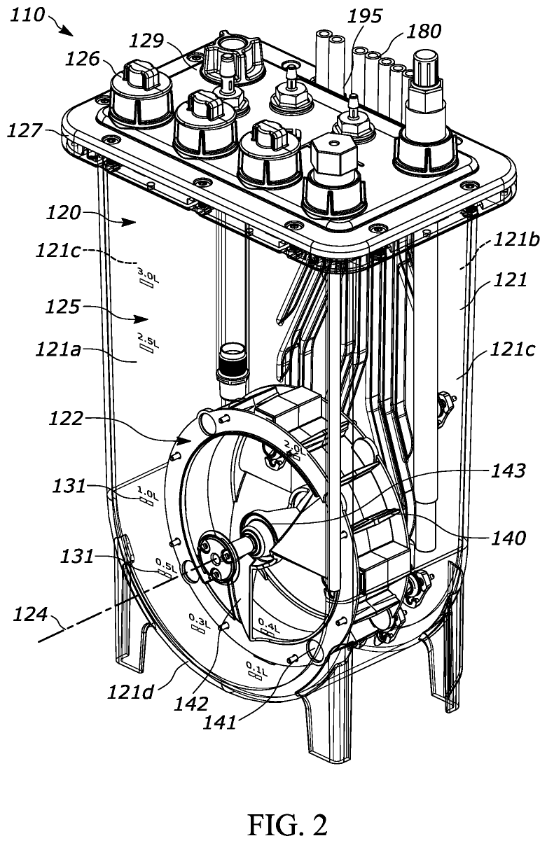

Turning now to FIG. 2, one example of a multi-level port vessel 110 for use in a bioreactor system and constructed in accordance with the teachings and principles of the present disclosure is illustrated. The vessel 110 in the illustrated embodiment includes a vertically oriented mixing wheel 122. In other embodiments, the multi-level port principles of the disclosure are equally applicable to a vessel having a non-vertically oriented mixing wheel (such as, for example, an angled mixing wheel or a horizontal mixing wheel (e.g., a spinner wheel)). Furthermore, in yet other embodiments, no mixing wheel may be present, for example in a tilted table vessel or bioreactor. The vessel 110 of FIG. 2 includes a vessel body 120 having outer walls 121 that define an interior volume 125. In the illustrated embodiment, the interior volume comprises approximately 4 L, with marking indicium 131 that indicate certain levels of the vessel body 120 that correspond to the indicated volumes. For example, in the illustrated embodiment, the indicium 131 indicate levels of 0.1 L, 0.3 L, 0.4 L, 0.5 L, 1.0 L, 2.0 L, 2.5 L, and 3.0 L. In other embodiments, other volumes may be indicated by the indicium 131. In other embodiments, the interior volume may comprise any volume including, for example, 0.5 L, 1 L, 2 L, 3 L, 10 L, 50 L, 100 L, 300 L, 500 L, or any volume as may be desirable for any given application for research, development, and/or commercial manufacturing interests. In yet other embodiments, the indicium may indicate percentages of full volume. For example, in the illustrated embodiment, if 3.0 L were the full usable volume, the indicium could indicate approximate percentages of full volume, e.g., 3%, 9%, 12%, 15%, 33%, 66%, 83%, and 100%. Other volumes percentages are also possible to meet the desired needs. For example, percentages in other embodiments may be labeled 10%, 20%, 30%, 40%, 50%, 60%, 70%, 80%, 90%, and/or 100% or any combination thereof. In yet other embodiments, any combination of percentages may be labeled in any value to meet the requirements for a user.

Cells and excipient (or other fluid mixtures, media, gases, solids, etc.) may be held in the interior volume 125. The vessel body 120 may include a front wall 121a, a rear wall 121b, and a pair of side walls 121c. Additionally, the illustrated embodiment includes a curved bottom wall 121d and an open top 127 that is covered by a removable lid 129. In other embodiments, the outer walls 121 may form other shapes, such as cylindrical, prismatic, or other non-defined shapes, as needed to best accomplish the desired cell growth or chemical reactions. Although the bottom wall 121d is illustrated as being curved, in other embodiments, the bottom wall 121d may be flat, or angled.

The vertically oriented wheel 122 is enclosed within the vessel body 120 for maintaining cells in suspension and/or for maintaining a homogeneous mixture within the interior volume 125. As used herein, a vertically oriented wheel 122 is defined as a wheel having a horizontal rotation axis so that the wheel rotates in a vertical plane when the vessel is placed on a horizontal surface. The vertically oriented wheel 122 is positioned in a lower portion of the vessel body 120 and oriented in a vertical plane and rotates about a horizontal axis 124. In some embodiments, a center line around which the curve of the bottom wall 121d of the vessel body 120 is formed resides coaxially with the horizontal axis 124 of the vertically oriented wheel 122. In some processes, cells and excipient may be introduced into the vessel by removing a threaded port cap 126 on the removable lid 129 and pipetting or pouring the cells and excipient through an opening or port in the removable lid 129. The cap 126 may be threaded back onto the port to seal prior to cell dispensing to minimize the potential for introducing foreign materials.

The vertically oriented wheel 122 may include any type of wheel and, in one version, includes a plurality of paddles 140 along its outer periphery that generate a sweeping motion of the fluid within the interior volume 125 during rotation to counteract cell settling in the excipient. The paddles 140, may include permanent magnets 141, which are used to couple with magnets on the agitation controller (not shown) to drive the rotation of the vertically oriented wheel 122. The vertically oriented wheel 122 may also include opposed vanes 142 extending from the paddles 140 to an inner hub 143 that create bi-axial fluid flow as the vertically oriented wheel 122 rotates.

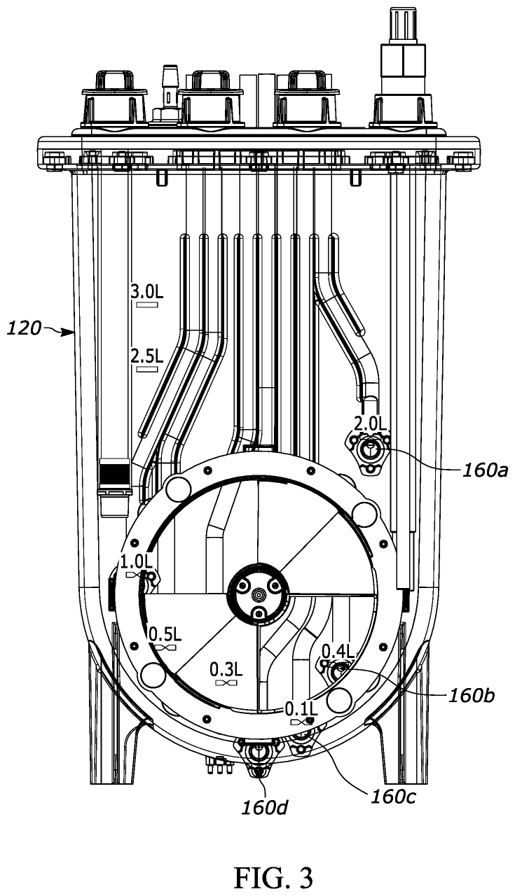

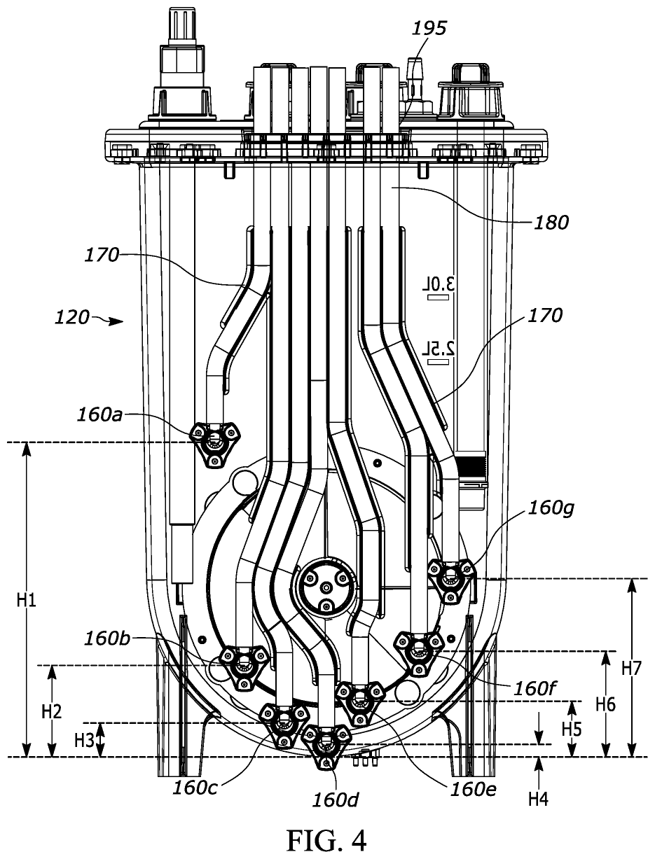

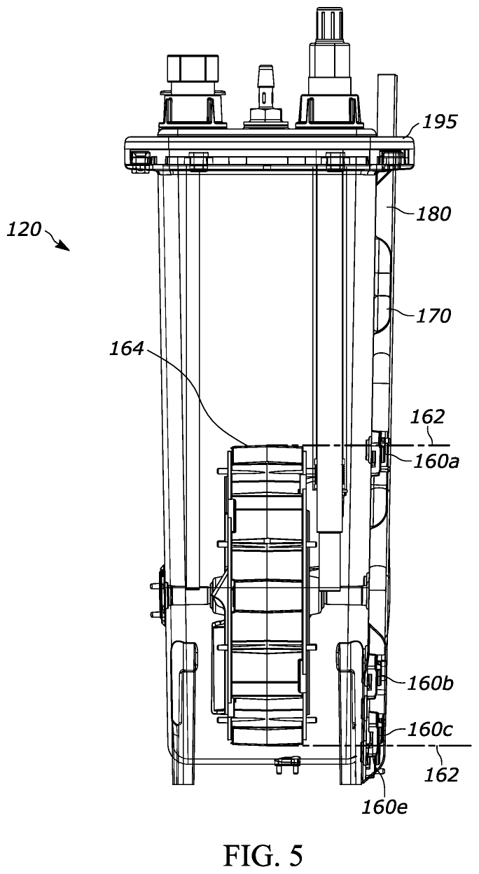

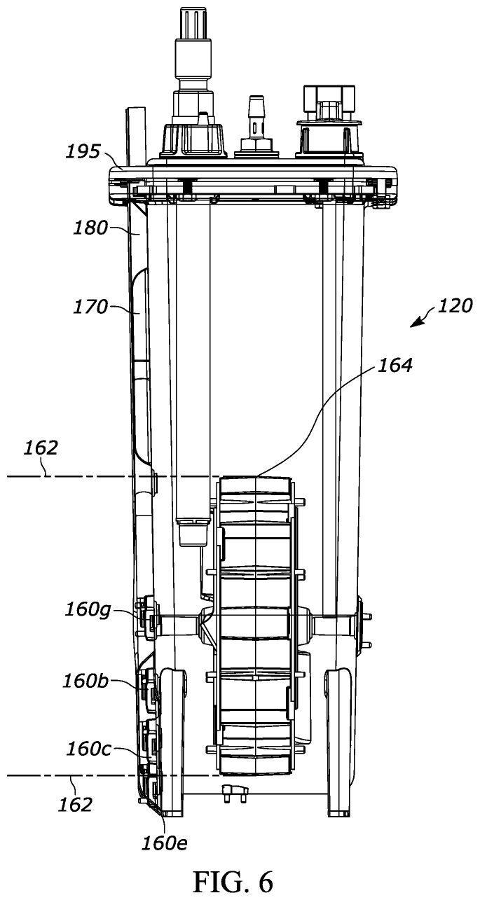

Turning now to FIGS. 3-6, a plurality of ports 160a-g (seen best in FIG. 4) are disposed in the rear wall 121b of the vessel body 120. In other embodiments, any one or more individual ports 160a-g of the plurality of ports 160a-g may be disposed in one or more of the rear wall 121b, the front wall 121a, or the side walls 121c. A projection 162 of a perimeter 164 of the vertically oriented mixing wheel 122 is illustrated in FIGS. 5 and 6. The upper and lower projections 162 of the perimeter 164 of the vertically oriented mixing wheel 122 illustrate the boundaries of the elevations within the interior volume 125 that are occupied at least partially by the vertically oriented mixing wheel 122, and which are not accessible to pipettes or sensors extending into the interior volume 125 through the removable lid 129. At least one port 160a-g in the plurality of ports 160a-g is located at an elevation that is at least partially encompassed by the projection 162 of the perimeter 164 of the vertically oriented mixing wheel 122.

In the illustrated embodiment, the plurality of ports 160a-g comprises two or more ports, more specifically in the illustrated embodiment, the plurality of ports 160a-g comprises seven ports 160a-g. As can be seen in FIG. 4, a first port 160a is located at an elevation at least partially above the projection 162 of the perimeter 164 of the vertically oriented mixing wheel. A second port 160b is located at an elevation that is at least partially encompassed by the projection 162 of the perimeter 164 of the vertically oriented mixing wheel 122. A third port 160c is located at an elevation at least partially below the projection 162 of the perimeter 164 of the vertically oriented mixing wheel 122.

In the illustrated embodiment, the first port 160a is positioned at a first height H1 from the bottom of the vessel body 120 and above the second port 160b, the first height H1 of the first port 160a corresponding to a vertical dimension of a volume of at least 2.0 L (or 66% of the full usable volume) in the interior volume 125 of the vessel body 120. The second port 160b is positioned at a second height H2 from the bottom of the vessel body 120 and above the third port 160c, the second height of the second port 160b corresponding to a vertical dimension of a volume of at least 0.4 L (or 12% of the full usable volume) in the interior volume 125 of the vessel body 120. The third port 160c is positioned at a third height H3 from the bottom of the vessel body 120 and corresponding to a vertical dimension of a volume of at least 0.1 L (or 3% of the full usable volume) in the interior volume 125 of the vessel body 120.

Other ports may be located at other locations. For example, a fourth port 160d may be positioned at a fourth height H4 from the bottom of the vessel body 120 corresponding to approximately 1% or less of the full usable volume of the vessel body 120. A fifth port 160e may be positioned at a fifth height H5 from the bottom of the vessel body 120 corresponding to approximately 0.3 L (or 9% of the full usable volume) in the interior volume 125 of the vessel body 120. A sixth port 160f may be positioned at a sixth height H6 from the bottom of the vessel body 120 corresponding to approximately 0.5 L (or 15% of the full usable volume) in the interior volume 125 of the vessel body 120. A seventh port 160g may be positioned at a seventh height H7 from the bottom of the vessel body corresponding to approximately 1.0 L (or 33% of the full usable volume) in the interior volume 125 of the vessel body 120.

In other embodiments, the ports 160 may have different relative positions and/or correspond to other interior volumes. For example, the ports may correspond to internal volumes of approximately 0.1 L (3%), approximately 0.3 L (9%), approximately 0.4 L (12%), approximately 0.5 L (15%), approximately 1.0 L (33%), approximately 2.0 L (66%), approximately 2.5 L (83%), and approximately 3.0 L (100%). The term “approximately” when used herein means that the recited term encompasses plus or minus 10% of the recited value. In other embodiments, the ports could be placed at any combination of volumes or percentages of full volume. For example, the foregoing description relates to the vessel body depicted in FIG. 4, but the disclosure applies to any vessel body having any total volume. As such, the locations of the ports may correspond to percentages of full volume, regardless of the associated partial volumes. For example, regardless of the full usable volume, the ports could be located at approximate percentages of full volume, e.g., 3%, 9%, 12%, 15%, 33%, 66%, 83%, and 100%. Other volumes percentages are also possible to meet the desired needs. For example, the ports in other embodiments may be located at 10%, 20%, 30%, 40%, 50%, 60%, 70%, 80%, 90%, and/or 100% of the full usable volume or any combination thereof. In yet other embodiments, the ports can be located at any combination of percentages to meet requirements for a user.

A tube guide 170 may be located proximate to at least one port 160a-g. The tube guide 170 comprises a pair of outwardly extending ribs extending parallel to each other and defining a channel configured to receive a tube 180 that is connected to at least one port 160a-g for providing access to the interior volume 125 via at least one port 160a-g. The channels defined by the tube guides 170 may be entirely linear, may include different linear portions, may include curved portions, or may be otherwise configured and designed to maintain the tubes in a tidy configuration. In the illustrated embodiment, a plurality of tube guides 170 is disposed proximate to the plurality of ports 160a-g, each tube guide 170 of the plurality of tube guides 170 being located proximate to a different port 160a-g in the plurality of ports 160a-g. Each tube guide 170 has a width dimensioned to frictionally engage and retain the corresponding tube 180. The tube guides 170 are integrally formed with the remainder of the vessel. The tubes 180 can be constructed of a silicone material, for example, that possesses some resiliency such that the width or radial dimension of the tube 180 slightly compresses upon introduction into the tube guide 170 and naturally expands to apply a force against the inner walls for retaining the tube 180 in position. Other forms of tube guides are contemplated including tube guides that are not integrally formed with the vessel, tube guides that include clips or other mechanical retention mechanisms, and any other configuration capable of retaining the tubes in close proximity to the vessel wall.

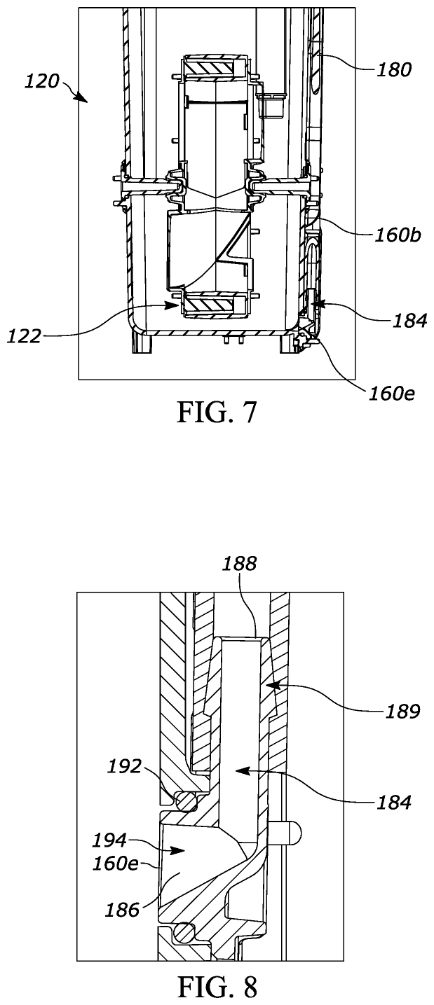

Turning now to FIGS. 7 and 8, each port 160a-g may be fluidly connected to a molded elbow 184, which is secured to a corresponding tube 180. The molded elbow 184 may have an inlet 186 and an outlet 188. The outlet 188 may include a barbed end 189 for securing to the tube 180. An o-ring 192 may be disposed between the molded elbow 184 and the port 160a-g to seal the molded elbow 184 against the vessel body 120. The molded elbow 184, in the depicted embodiment, comprises an angled cone 194 inlet so that a portion of the angled cone 194 is not perpendicular to the port 160a-g. In other words, the angled cone 194 allows gravity to direct fluid that may otherwise reside in the elbow 184 into the interior volume of the vessel, so that fluid is unable to pool in the molded elbow 184. While the elbow 184 has been described as including an angled cone 194 for this purpose, other embodiments may simply include an angled planar surface (or other angled or contoured surface) at the bottom of the inlet 186 portion to achieve a similar objective under the force of gravity.

Returning now to FIGS. 2, 4, 5, and 6, the removable lid 129 comprises a cut-out portion 195 having at least one recess configured to receive at least one of the tubes 180 connected to the ports 160. The cut-out portion 195 advantageously collects and secures the tubes 180 within the outer perimeter of the removable lid 129, which results in a compact and secure arrangement.

The multi-level port vessel described above advantageously allows access to the interior volume 125 of the vessel body 120 from any elevation within the vessel body 120, even elevations occupied at least in part by the mixing wheel 122. The interior volume 125 of the vessel body 120 may be accessed through the ports 160a-f located in the rear wall 121b (or the side walls 121c, or the front wall 121a). Elevations at least partially occupied in part by the mixing wheel 122 are accessible through the ports located within the projection 162 of the perimeter 164 of the mixing wheel 122, as described above. The interior volume 1215 may be simultaneously accessed through multiple ports at multiple elevations, at the same time or at different times, and while the mixing wheel is rotating or stopped.

The interior volume 125 may be accessed for the purpose of extracting fluid from the interior volume 125 via the port 160a-g. In other examples, accessing the interior volume 125 may include penetrating the port with an extraction tool for the extraction of fluid from within the interior volume 125, or the accessing may include penetrating the port with an introducer tool to add media to the interior volume 125. In such versions, extraction tools and/or introducer tools may be flexible tools designed to be threaded through one of the plurality of tubes 180 and into the vessel via the corresponding port 160a-g, for example.

Sensors may also be introduced into the interior volume 125 though the ports 160a-g so that the sensors are located at the desired level in the vessel body 120. Some examples of sensors that may be introduced through the ports 160a-g include temperature sensors, pH sensors, concentration sensors, pressure sensors, etc. In such versions, the sensors may be carried by flexible elongated members designed to be threaded through one of the plurality of tubes 180 and into the vessel via the corresponding port 160a-g, for example.

Accessing the interior volume 125 may also include adding fluid, gas, cells, buffer, and/or other media to the interior volume 125 of the vessel body 120 through the port(s) 160a-g.

Any of the accessing discussed above may be accomplished while the mixing wheel 122 is rotating due to the location of the ports 160a-g in the vertical walls of the vessel body 120. As such, access is granted along a horizontal axis of the vessel body 120. This can be advantageous for taking sample, introducing media, and/or sensing conditions in the vessel because those conditions change depending on whether the mixing wheel is rotating or stopped. The configuration of the ports of the present application as such advantageously allow users to gain access to the vessel in a manner that does not interfere with the rotation of the mixing wheel.

When accessing the interior volume 125 through multiple ports 160a-g, the access may include using ports 160a-g having locations that are at an elevation that is (i) occupied at least partially by a projection of the perimeter of the mixing wheel, (ii) at least partially above a projection of the perimeter of the mixing wheel, and/or (iii) at least partially below a projection of the perimeter of the mixing wheel 122.

While various embodiments have been described herein, it will be understood that modifications may be made thereto that are still considered within the scope of the appended claims.

1. A multi-level port vessel, comprising:
a vessel body defining an interior volume, the vessel body having a front wall, a rear wall, two side walls, a bottom, and a top, the top being open and being adapted to be covered by a removable lid;
a vertically oriented mixing wheel disposed within the interior volume, the vertically oriented mixing wheel having a substantially horizontal rotation axis when the vessel body is disposed on a substantially horizontal surface;
a plurality of ports disposed in one of the rear wall, the front wall, or the side walls, at least one port of the plurality of ports being located at an elevation that is at least partially encompassed by a projection of the perimeter of the vertically oriented mixing wheel.

2. The vessel of claim 1, wherein the plurality of ports comprises two or more ports.

3. The vessel of claim 1, wherein the plurality of ports comprises a first port located at an elevation at least partially above the perimeter of the vertically oriented mixing wheel, a second port being located at an elevation that is at least partially encompassed by the perimeter of the vertically oriented mixing wheel, and a third port being located at an elevation at least partially below the perimeter of the vertically oriented mixing wheel.

4. The vessel of claim 3, wherein the first port is positioned at a first height from a bottom of the vessel body, above the second port, the first height corresponding to a vertical dimension of a volume of at least 66% of a full usable volume of the interior volume of the vessel body.

5. The vessel of claim 3, wherein the second port is positioned at a second height from a bottom of the vessel body, above the third port, the second height corresponding to a vertical dimension of a volume of approximately 12% of a full usable volume of the interior volume of the vessel body.

6. The vessel of claim 1, further comprising a tube guide proximate the at least one port.

7. The vessel of claim 6, wherein the tube guide comprises a pair of outwardly extending ribs that are configured to receive a tube that is connected to the at least one port for providing access to the interior volume via the at least one port.

8. The vessel of claim 6, further comprising a plurality of tube guides, each tube guide of the plurality of tube guides being located proximate to a different port in the plurality of ports.

9. The vessel of claim 6, wherein the at least one port is fluidly connected to a molded elbow, the molded elbow having an inlet and an outlet, the outlet secured to the tube.

10. The vessel of claim 9, further comprising an o-ring between the molded elbow and the port.

11. The vessel of claim 9, wherein the inlet of the molded elbow comprises an angled cone so that a portion of the angled cone is not perpendicular to the port.

12. The vessel of claim 1, further comprising a removable lid covering the top of the vessel.

13. The vessel of claim 12, wherein the removable lid comprises at least one of (i) a cut-out portion having at least one recess configured to receive a tube connected to the at least one port, and (ii) at least one inlet opening.

14. (canceled)

15. The vessel of claim 1, wherein the vertically oriented mixing wheel comprises at least one of (i) a plurality of mixing paddles, and (ii) a plurality of magnets.

16. (canceled)

17. The vessel of claim 1, wherein the at least one port is located in the rear wall of the vessel.

18. The vessel of claim 17, wherein another port is located in the front wall or in one of the side walls of the vessel.

19. A method of accessing an interior volume of a vessel, the method comprising:
providing a vessel body defining an interior volume, the vessel body including a vertically oriented mixing wheel having a substantially horizontal rotation axis when the vessel body is disposed on a substantially horizontal surface; and
accessing the interior volume of the vessel body via one of a plurality of ports in the vessel body, each port being located in one of a rear wall, a front wall, or a side wall of the vessel body, wherein at least one of the plurality of ports is located at an elevation that is occupied at least partially by a projection of the perimeter of the mixing wheel.

20. The method of claim 19, wherein accessing the interior volume comprises extracting fluid from the interior volume via the port.

21. The method of claim 19, further comprising penetrating the port with an extraction tool.

22. The method of claim 19, wherein accessing the interior volume comprises adding fluid, gas, cells, buffer, and/or other media to the interior volume of the vessel body through the port.

23. The method of claim 19, further comprising penetrating the port with an introducer tool.

24. The method of claim 19, wherein accessing the interior volume comprises passing a sensor device through the port and into the interior volume of the vessel body.

25. The method of claim 19, further comprising rotating the mixing wheel about its horizontal axis, and wherein accessing the interior volume of the vessel body comprises accessing the interior volume while the mixing wheel is rotating.

26. The method of claim 19, wherein accessing the interior volume of the vessel body comprises accessing the interior volume along a horizontal axis.

27. The method of claim 19, wherein accessing the interior volume of the vessel body comprises accessing the interior volume at a location that is at an elevation that is (i) occupied at least partially by a projection of the perimeter of the mixing wheel, (ii) at least partially above a projection of the perimeter of the mixing wheel, or (iii) at least partially below a projection of the perimeter of the mixing wheel.
